SEQUENCE LISTING (2 sequences)

SEQ ID NO: 1 - protein, 31 aa, synthetic construct
  MOD_RES 2: note = X is alpha-aminoisobutyric acid (Aib)
  REGION 20: note = Lysine is attached to a C-18 fatty diacid through a PEG modified glutamic acid hydrophilic spacer
HXEGTFTSDVSSYLEGQAAKEFIAWLVRGRG

SEQ ID NO: 2 - protein, 31 aa, synthetic construct
  REGION 20: note = Lysine is attached to a C-16 fatty acid (palmitic acid) through a glutamic acid spacer
HAEGTFTSDVSSYLEGQAAKEFIAWLVRGRG

(19) United States
(12) Patent Application Publication
(10) Pub. No.: US 2023/0406900 A1
(43) Pub. Date: Dec. 21, 2023

(54) PROCESS FOR PREPARING GLUCAGON-LIKE PEPTIDE-1

(71) Applicant: ScinoPharm Taiwan, Ltd., Tainan (TW)

(72) Inventors: Ming-Chih Wu, Tainan (TW); Meng-Fen Ho, Tainan (TW); Tsung-Yu Hsiao, Tainan (TW)

(21) Appl. No.: 18/318,869

(22) Filed: May 17, 2023

Related U.S. Application Data
(60) Provisional application No. 63/348,003, filed on Jun. 1, 2022.

(51) Int. Cl. C07K 14/605; C07K 14/00

(52) U.S. Cl. CPC C07K 14/605; C07K 14/001

(57) ABSTRACT

The present disclosure provides an improved process for the preparation of glucagon-like peptide-1 agonist peptide. Specifically, it relates to a manufacturing process useful for reducing impurities and increasing the yields and the balance of cost in the preparation of liraglutide or semaglutide.

### CROSS-REFERENCES TO RELATED APPLICATIONS

This application claims priority to U.S. Provisional Application No. 63/348,003 filed Jun. 1, 2022, which is incorporated herein in its entirety for all purpose.

### REFERENCE TO A “SEQUENCE LISTING,” A TABLE, OR A COMPUTER PROGRAM LISTING APPENDIX SUBMITTED ON A COMPACT DISK

This application includes an electronically submitted sequence listing in .xml format. The .xml file contains a sequence listing entitled “4837-009US” for reference—“4837-009US” created on Sep. 4, 2023 and is 4,185 bytes in size. The sequence listing contained in this .xml file is part of the specification and is hereby incorporated by reference herein in its entirety.

### BACKGROUND OF THE INVENTION

Human glucagon-like peptide-1 (hereinafter referred to as GLP-1) receptor agonists include exenatide, liraglutide, dulaglutide, lixisenatide, and semaglutide. Among them, semaglutide (trade name Ozempic®) and liraglutide (trade name Victoza®), are indicated as an adjunct to diet and exercise to improve glycemic control in adults with type 2 diabetes mellitus.

Semaglutide is a peptide having SEQ ID NO: 1, which has been engineered to be 94% homologous to native human GLP-1 by substituting alpha-aminoisobutyric acid (Aib) for alanine at position 8 and arginine for lysine at position 34. Semaglutide is further derived from the noted engineered peptide by attaching a C-18 fatty diacid through a PEG modified glutamic acid hydrophilic spacer at the lysine residue of position 26. Accordingly, the structure of semaglutide with noted modifications based on native human GLP-1 is shown below:

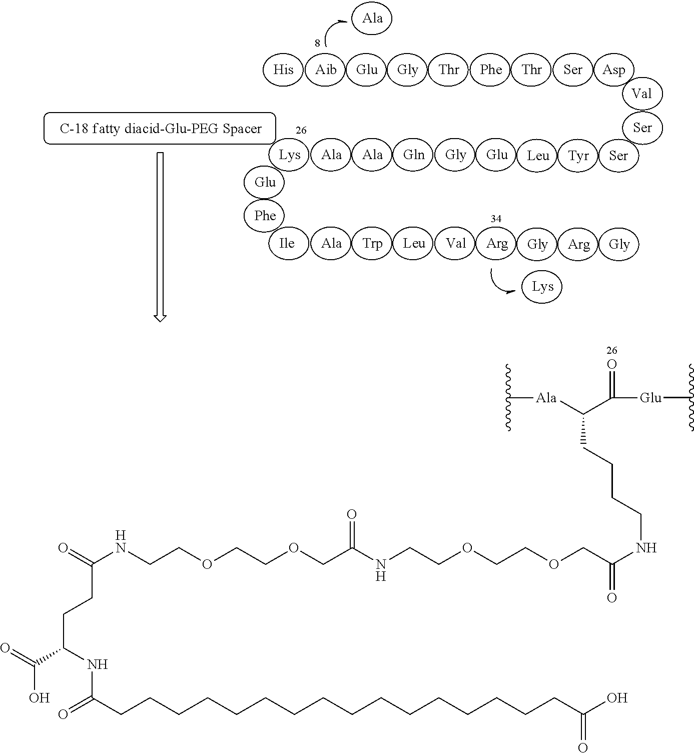

Semaglutide has a molecular formula of C₁₈₇H₂₉₁N₄₅O₅₉ and a molecular weight of 4,113.58 Daltons. It was originally developed by Novo Nordisk and approved by the U. S. Food and Drug Administration (FDA) in December 2017 under the trade name “Ozempic®”.

Liraglutide is a peptide having SEQ ID NO: 2, which has been engineered to be 97% homologous to native human GLP-1 by substituting arginine for lysine at position 34. Liraglutide is further derived from the noted engineered peptide by attaching a C-16 fatty acid (palmitic acid) through a glutamic acid spacer at the lysine residue of position 26. Accordingly, the structure of liraglutide with noted modifications based on native human GLP-1 is shown below:

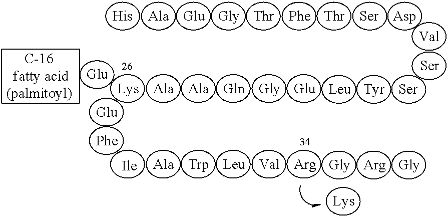

Liraglutide has a molecular formula of C₁₇₂H₂₆₅N₄₃O₅₁ and a molecular weight of 3,751.2 Daltons. It was originally developed by Novo Nordisk and approved by the U. S. Food and Drug Administration (FDA) in January 2010 under the trade name “Victoza®”.

Solid Phase Peptide Synthesis (SPPS) is relatively simple and useful for synthesis of peptides. An amino acid is attached to a solid phase by a linking group on the acid side, and to a protecting group on the amine side. The protecting group can be removed so that the second amino acid can be coupled to the amine side on the original amino acid. The second (and succeeding) amino acids are also initially protected, and thus the general process is to deprotect (protecting group), couple, and repeat the cycle until the desired peptide is completed, following which the completed peptide is cleaved from the solid phase.

U.S. Pat. No. 9,040,480B2 discloses a method of manufacturing a GLP-1 or GLP-1 agonist peptide, wherein using amino acid and pseudoproline dipeptides to synthesize the peptide on a solid phase but does not disclose the synthetic process for side chain of Lys for liraglutide or semaglutide.

China Patent No. 109311961B discloses a method for synthesizing semaglutide. The method comprises: coupling dipeptide fragments and tripeptide fragments with an amino acid with N-terminus Fmoc protection to obtain semaglutide but does not disclose the pseudoproline dipeptides used in the preparation of semaglutide.

China Patent Application Publication No. 110922470A discloses a method of manufacturing semaglutide, wherein sequentially coupling Fmoc-AEEA-OH, Fmoc-AEEA-OH and (γ-Glu-OtBu)-Octadecanedioic acid mono-tert-butyl ester for side chain of Lys but does not disclose any specific embodiments in the specification for semaglutide.

Despite the above-described manufacturing processes, there remains a need for the development of more efficient and improved processes for preparing GLP-1 agonist peptides. The present disclosure addresses this need and provides related advantages as well.

### SUMMARY OF THE INVENTION

In one aspect, the present disclosure provides a process for the preparation of glucagon-like peptide-1 agonist peptide represented by formula (1),

(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH

wherein:
X is Ala or Aib;
R is -γ-Glu-Hexadecanoic acid or -AEEA-AEEA-γ-Glu-Octadecanedioic acid; comprising steps of:
(a) synthesizing a protected peptide on a solid phase by stepwise coupling of a protected amino acid and a protected dipeptide, the protected dipeptide comprising a pseudoproline dipeptide and a non-pseudoproline dipeptide;
(b) cleaving the protected peptide from the solid phase, and deprotecting the protected peptide.

In another aspect, the present disclosure provides a glucagon-like peptide-1 agonist peptide represented by formula (1),

(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH

wherein:
X is Ala or Aib;
R is -γ-Glu-Hexadecanoic acid or -AEEA-AEEA-γ-Glu-Octadecanedioic acid; and
a content of deletion of X at position 2 of the glucagon-like peptide-1 agonist peptide is less than 0.10%.

In another aspect, the present disclosure provides a glucagon-like peptide-1 agonist peptide obtained according to a process for the preparation of glucagon-like peptide-1 agonist peptide represented by formula (1),

(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH

wherein:
X is Ala or Aib;
R is -γ-Glu-Hexadecanoic acid or -AEEA-AEEA-γ-Glu-Octadecanedioic acid; comprising steps of:
(a) synthesizing a protected peptide on a solid phase by stepwise coupling of a protected amino acid and a protected dipeptide, the protected dipeptide comprising a pseudoproline dipeptide and a non-pseudoproline dipeptide;
(b) cleaving the protected peptide from the solid phase, and deprotecting the protected peptide;
a content of deletion of X at position 2 of the glucagon-like peptide-1 agonist peptide is less than 0.10%.

The various features of novelty which characterize the invention are pointed out with particularity in the claims annexed to and forming a part of the disclosure. For a better understanding of the invention, its operating advantages, and specific objects attained by its use, reference should be had to the descriptive matter in which there are illustrated and described preferred embodiments of the invention.

### DETAILED DESCRIPTION OF PREFERRED EMBODIMENTS

### I. General

The present disclosure provides an improved process for the preparation of glucagon-like peptide-1 agonist peptide. Specifically, it relates to a manufacturing process useful for reducing impurities and increasing the yields and the balance of cost in the preparation of liraglutide or semaglutide. In particular, the processes utilize a pseudoproline dipeptide and a non-pseudoproline dipeptide in the preparation step, which provides better yields and less impurities for the preparation of liraglutide or semaglutide.

The following Table 1 summarizes the advantages or characteristics of the embodiments of the present disclosure compared with the processes reported in the art.

TABLE 1

| | Reference's | | |
|---|---|---|---|
| | technology relevant to | Embodiments of this | Advantage of embodiments |
| Reference | this invention | invention | of this invention |
| U.S. Pat. No. | Using Fmoc-Val- | Using pseudoproline | Des-Aib impurity could be |
| 8,445,433B2 | Ser(Psi^{(Me, Me)}pro)-OH | dipeptide Fmoc-Val- | significantly reduced by |
| | as material for ValSer | Ser(Psi^{(Me, Me)}pro)-OH as | using non-pusedoproline |
| | coupling | material for ValSer coupling, | dipeptide (HisAib) as |
| | | and non-pusedoproline | material which could also |
| | | dipeptide for other amino | improve the total conversion |
| | | acids coupling | rate of Aib² and His¹ reaction |
| CN109311961B | Using a combination | Using pseudoproline | 1. The usage of dipeptide |
| | of dipeptide and | dipeptide Fmoc-Val- | strategy is far much less |
| | tripeptide as starting | Ser(Psi^{(Me, Me)}pro)-OH as | complicated than the prior art |
| | materials for synthesis | material for ValSer coupling, | 2. The reaction efficiency of |
| | | and non-pusedoproline | deFmoc, coupling with Asp⁹ |
| | | dipeptide for other amino | to His¹, and grafting could be |
| | | acids coupling | improved by using |
| | | | pseudoproline dipeptide as a |
| | | | starting material which |
| | | | results in 5-10% |
| | | | improvement in purity and |
| | | | assay of crude. |
| CN110922470A | Sequentially coupling | Using pseudoproline | As mentioned above, |
| | Fmoc-AEEA-OH, | dipeptide Fmoc-Val- | pseudoproline dipeptide and |
| | Fmoc-AEEA-OH and | Ser(Psi^{(Me, Me)}pro)-OH as | non- pseudoproline dipeptide |
| | (γ-Glu-OtBu)- | material for ValSer coupling, | could improve the |
| | Octadecanedioic acid | and non-pusedoproline | production efficiency and |
| | mono-tert-butyl ester | dipeptide for other amino | reduce the certain critical |
| | for side chain | acids coupling | impurities. |

### II. Definitions

SEQ ID NO: 1 refers to the amino acid sequence of the engineered peptide in semaglutide. The sequence is represented by: H-His-Aib-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly-OH (SEQ ID NO: 1).

SEQ ID NO: 2 refers to the amino acid sequence of the engineered peptide in liraglutide. The sequence is represented by: H-His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Arg-Gly-Arg-Gly-OH (SEQ ID NO: 2).

### III. Description of the Embodiments

For the purpose of clarity and as an aid in the understanding of the disclosure, the terms and abbreviations used in the specification and claims are defined in Table 2.

TABLE 2
The terms and abbreviations used in the specification and claims

| | |
|---|---|
| ACN | acetonitrile |
| Ac₂O | acetic anhydride |
| AEEA | 2-(2-(2-Aminoethoxy)ethoxy)acetic acid |
| Aib | α-aminoisobutyric acid |
| Ala | alanine |
| Alloc | allyloxycarbonyl |
| Arg | arginine |
| Asn | asparagine |
| Asp | aspartic acid |
| Boc | tert-butyloxycarbonyl |
| Cbz | carboxybenzyl |
| DBU | diazabicyclo[5.4.0]undec-7-ene |
| DCHA | dicyclohexylamine |
| DCM | dichloromethane |
| DEPBT | 3-(diethoxy-phosphoryloxy)-3H-benzo[d][1,2,3]triazin- |
| | 4-one |
| DIC | N,N′-Diisopropylcarbodiimide |
| DIPEA | diisopropylethylamine |
| DMAP | 4-dimethylaminopyridine |
| DMF | N,N-dimethylformamide |
| DMSO | dimethyl sulfoxide |
| DTE | dithioerythriol |
| DTT | dithiothreitol |
| EDT | ethanedithiol |
| equiv. | equivalent |
| Fmoc | 9-fluorenylmethoxycarbonyl |
| Fmoc- | {2-[2-(Fmoc-amino)ethoxy]ethoxy}acetic acid |
| AEEA-OH | |
| Gln | glutamine |
| Glu | glutamic acid |
| Gly | glycine |
| HATU | 1-[bis(dimethylamino)methylene]-1H-1,2,3-triazolo[4,5- |
| | b]pyridinium 3-oxid hexafluorophosphate |
| HBTU | 1-[bis(dimethylamino)methylene]-1H-benzotriazolium |
| | 3-oxide hexafluorophosphate |
| His | histidine |
| HOBt•H₂O | 1-hydroxybenzotriazole monohydrate |
| HPLC | high performance liquid chromatography |
| Ile | isoleucine |
| IPE | isopropyl ether |
| ivDde | 1-(4,4-dimethyl-2,6-dioxocyclohex-1-ylidene)-3- |
| | methylbutyl |
| Leu | leucine |
| Lys | lysine |
| MeOH | methanol |
| Mmt | 4-methoxytrityl |
| MTBE | methyl tert-butyl ether |
| Mtt | 4-methyltrityl |
| MW | molecular weight |
| NMP | N-methylpyrrolidone |
| OMpe | 3-methylpent-3-yl ester |
| OtBu | tert-butoxy |
| Pbf | 2,2,4,6,7-pentamethyl-2,3-dihydrobenzofuran-5-sulfonyl |
| Pd(PPh₃)₄ | tetrakis(triphenylphosphine)palladium |
| Phe | phenylalanine |
| Pmc | 2,2,5,7,8-pentamethylchroman-6-sulfonyl |
| PPW | purified process water |
| Ser | serine |
| SPPS | solid phase peptide synthesis |
| Ste | octadecanedioic acid |
| TATU | 1-[bis(dimethylamino)methylene]-1H-1,2,3-triazolo[4,5- |
| | b]pyridinium 3-oxide tetrafluoroborate |
| TBTU | 1-[bis(dimethylamino)methylene]-1H-benzotriazolium |
| | 3-oxide tetrafluoroborate |
| tBu | tert-butyl |
| TFA | trifluoroacetic acid |
| TES | triethylsilane |
| THF | tetrahydrofuran |
| TIPS | triisopropylsilane |
| Trp | tryptophan |
| Trt | trityl |
| Val | valine |

In a first aspect, the present invention provides a process for the preparation of glucagon-like peptide-1 agonist peptide represented by formula (1),

(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH

wherein:
X is Ala or Aib;
R is -γ-Glu-Hexadecanoic acid or -AEEA-AEEA-γ-Glu-Octadecanedioic acid; comprising steps of:
(a) synthesizing a protected peptide on a solid phase by stepwise coupling of a protected amino acid and a protected dipeptide, the protected dipeptide comprising a pseudoproline dipeptide and a non-pseudoproline dipeptide;
(b) cleaving the protected peptide from the solid phase, and deprotecting the protected peptide to obtain the peptide.

In some embodiments, the pseudoproline dipeptide is Fmoc-Val-Ser(Psi^{(Me,Me)}pro)-OH. Fmoc-Val-Ser(Psi^{(Me,Me)}pro)-OH is used in the synthesis of liraglutide or semaglutide, which can reduce the Des-Thr⁵ impurity and increase the yield.

In some embodiments, the non-pseudoproline dipeptide is selected from the group consisting of Boc-His(Boc)-Aib-OH, Boc-His(Boc)-Ala-OH, Boc-His(Trt)-Aib-OH, and Boc-His(Trt)-Ala-OH. Boc-His(Boc)-Aib-OH, Boc-His(Boc)-Ala-OH, Boc-His(Trt)-Aib-OH or Boc-His(Trt)-Ala-OH is used in the synthesis of liraglutide or semaglutide, which can increase the purity of the crude peptide. Preferably, the non-pseudoproline dipeptide is selected from the group consisting of Boc-His(Trt)-Aib-OH and Boc-His(Trt)-Ala-OH. Boc-His(Trt)-Aib-OH or Boc-His(Trt)-Ala-OH is used in the synthesis of liraglutide or semaglutide, which can reduce the Des-Ala e or Des-Aib² impurity and increase the yield (conversion rate). In particular, the pseudoproline dipeptide, such as Fmoc-Val-Ser(Psi^{(Me,Me)}pro)-OH and the non-pseudoproline dipeptide, such as Boc-His(Trt)-Aib-OH or Boc-His(Trt)-Ala-OH, which are used together in the synthesis of liraglutide or semaglutide, have the synergistic effect that can dramatically reduce the Des-Thr⁵, Des-Gly⁴, Des-Ala² or Des-Aib² impurities and increase the yield.

In some embodiments, the coupling reagent DEPBT/DIPEA is used in the coupling of non-pseudoproline dipeptide Boc-His(Trt)-Aib-OH or Boc-His(Trt)-Ala-OH. The coupling reagent DEPBT/DIPEA is used in the coupling of Boc-His(Trt)-Aib-OH or Boc-His(Trt)-Ala-OH in the synthesis of liraglutide or semaglutide, which can increase the purity of the crude peptide and the yield.

In some embodiments, the non-pseudoproline dipeptide further comprises at least one selected from the group consisting of Boc-Arg(Pbf)-Gly-OH and Boc-Glu(OtBu)-Gly-OH.

In some embodiments, no more than 3 types of the non-pseudoproline dipeptide are used. Preferably, no more than 2 types of the non-pseudoproline dipeptide are used. Most preferably, no more than 1 type of the non-pseudoproline dipeptide is used.

In some embodiments, Fmoc-Lys(Alloc)-OH or Fmoc-Lys(ivDde)-OH is used as the protected amino acid of Lys.

In some embodiments, the peptide is liraglutide.

In some embodiments, the peptide is semaglutide.

In some embodiments, the R is -AEEA-AEEA-γ-Glu-Octadecanedioic acid, and using AEEA-AEEA-(γ-Glu-OtBu)-Octadecanedioic acid mono-tert-butyl ester to couple with the ε-amino side chain of the Lys after the step (a).

In some embodiments, the R is -AEEA-AEEA-γ-Glu-Octadecanedioic acid, and using Fmoc-AEEA-OH, Fmoc-AEEA-OH, (γ-Glu-OtBu)-Octadecanedioic acid mono-tert-butyl ester to sequentially couple with the ε-amino side chain of the Lys after the step (a).

In some embodiments, the process further comprises a step of purification after the step (b).

In some embodiments, the solid phase may be, but is not limited to, polystyrene based 4-alkoxybenzyl alcohol (Wang) resin, polymeric diphenyldiazomethane (PDDM) resin, 4-(2′,4′-dimethoxyphenyl-Fmoc-aminomethyl)-phenoxym-ethyl-polystyrene (Rink) resin, 2-methoxy-4-alkoxybenzyl alcohol (Sasrin) resin, and 2-chlorotrityl chloride (CTC) resin. Moreover, any other resin suitable for SPPS may be used.

In some embodiments, the protected amino acid is the amino acid comprising the N-terminal alpha amine protecting group. The N-terminal alpha amine protecting group may be, but is not limited to, Fmoc, Boc, and Cbz. Preferably the N-terminal alpha amine protecting group is Fmoc or Boc, and more preferably Fmoc. In any subsequent coupling step, the N-terminal alpha amine protecting group of the peptide formed in the preceding coupling step is removed, for example by reaction with a cleavage reagent, e.g. a base such as piperidine in the case of Fmoc, or an acid such as TFA in the case of Boc, before the next protected amino acid or protected dipeptide is coupled.

In some embodiments, the SPPS method is based on an Fmoc synthesis strategy. Fmoc protecting group is cleaved (deFmoc) with a cleavage reagent comprising a base in a solvent. The base may be selected from secondary amines, such as piperidine and 4-methyl piperidine. The solvent may be selected from the group consisting of DMF, NMP, DMSO, DCM, THF, acetonitrile, toluene, and mixtures thereof. The reaction is commonly carried out at ambient temperature, for example, within a temperature range of 15 to 30° C. The base-labile and acid-stable Fmoc can be cleaved off within a short period of time, such as 2 to 15 minutes.

In some embodiments, the cleavage reagent is selected from the group consisting of 5-50% (w/w) piperidine or 4-methyl piperidine in DMF, 5-50% (w/w) piperidine or 4-methyl piperidine in NMP, 1-5% (w/w) DBU in DMF, and 50% (w/w) morpholine in DMF. The cleavage reagent is washed out carefully after Fmoc removal. DMF is used for washing until neutral pH. To ensure complete base removal, it may be advantageous to add small amounts of HOBt in subsequent washing cycles.

In some embodiments, the protected amino acid is the amino acid further comprising the amine side chain protecting group. The amine side chain protecting group may be, but is not limited to Fmoc, Boc, Mtt, Mmt, Trt, Pbf, OtBu, tBu, OMpe, Pmc, ivDde, and Alloc.

In some embodiments, the protected amino acid may be, but is not limited to Fmoc-Aib-OH, Fmoc-Ala-OH, Fmoc-Arg(Pbf)-OH, Fmoc-Arg(Pmc)-OH, Fmoc-Asn(Trt)-OH, Fmoc-Asn(Mtt)-OH, Fmoc-Asp(tBu)-OH, Fmoc-Asp(OtBu)-OH, Fmoc-Asp(OMpe)-OH, Fmoc-Cys(Trt)-OH, Fmoc-Cys(Mmt)-OH, Fmoc-Gly-OH, Fmoc-Gln(Mtt)-OH, Fmoc-Gln(Trt)-OH, Fmoc-Glu(tBu)-OH, Fmoc-Glu(OtBu)-OH, Fmoc-His(1-Trt)-OH, Fmoc-Ile-OH, Fmoc-Leu-OH, Fmoc-Lys(Alloc)-OH, Fmoc-Lys(Boc)-OH, Fmoc-Lys(ivDde)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, and Boc-His(Boc)-OH.

In some embodiments, the synthesis of liraglutide or semaglutide which typically contains protected amino acid residues, is preferably carried out using an acid-labile N-terminal alpha amine protecting group in the His residue, such as Boc, so that the N-terminal alpha amine protecting group and the amine acid side chain protecting groups in the protected liraglutide or semaglutide sequence can be removed (optionally along with any solid phase, e.g. Wang resin) in one step. For example, the His N-terminal alpha amine Boc group may be removed together with the acid-labile amine side chain protecting groups and Wang resin by treatment with a cleavage cocktail (typically a cleavage cocktail comprises TFA and can be a mixture of TFA with DTT in DCM), thereby producing liraglutide or semaglutide.

In some embodiments, the coupling of the protected amino acid or protected dipeptide is carried out in DMF as a solvent, for example, the protected amino acid or protected dipeptide, a coupling reagent and optionally an additive are dissolved in DMF and mixed. DIC may be used as coupling reagent in combination with HOBt as an additive. Alternatively, TBTU or DEPBT may be used to convert the Fmoc amino acid into an active ester in the presence of a base, preferably DIPEA. The coupling reagent/additive mixture is selected from the group consisting of DIC/HOBt, TBTU/DIPEA, TATU/DIPEA, DEPBT/DIPEA, HBTU/DIPEA, and HATU/DIPEA. The coupling of the protected amino acid or protected dipeptide is carried out within a period of time, such as 1 to 74 hours. Optionally, it may be advantageous to perform one or more re-coupling steps in order to complete the conversion of amino groups.

The capping may be performed to block unreacted N-terminal alpha amines of peptide in the following steps of synthesis and avoid the formation of deletion variants. It may be achieved by a short treatment of the resin bound peptide with a large excess of a highly reactive unhindered acid derivative such as N-hydroxysuccinimide, acetic anhydride or benzoyl chloride, and a base such as pyridine, collidine, or DIPEA. The capping will typically yield a truncated sequence, which generally differs from the final peptide and can be completely separated. The reagents used in the capping step are typically filtered off and the resin bound peptide is carefully washed with DMF and optionally methanol, before proceeding to the next deprotection step.

In some embodiments, the removing of Alloc or ivDde protecting group of Lys from resin bound peptide may be carried out by the use of a catalyst such as [Pd(PPh₃)₄]=Pd(Ph₃P)₄, Pd₂(dba)₃·CHCl₃, Pd(dba)₂, Pd(Ph₃P)₂Cl₂, (Ph₃P)₂NiCl₂, or Pd(OAc)₂. Preferably, [Pd(PPh₃)₄] may be used.

For cleaving the peptide off the resin, a composition cmoprising TFA may be used. Preferably, the composition comprising more than 50% (v/v) TFA, more preferably more than 75% (v/v) TFA, in particular at least 80% (v/v) or even at least 90% (v/v) TFA. The composition may also comprise water and/or one or more scavengers. Preferably, the scavengers are thiol scavengers, such as EDT, DTT, and DTE; and silane scavengers, such as TIPS, and TES.

After cleaving the peptide from the resin, the resin is then separated, usually by filtration. An anti-solvent is uesd to mix with the obtained filtrate, then the crude peptide is precipitated. The anti-solvent may be selected from the group consisting of diethyl ether, IPE, MTBE, ACN, and mixtues thereof.

The obtained crude peptide can be subjected to further purification by one or more chromatographic methods. The chromatographic method comprises gel permeation chromatography (GPC), size exclusion chromatography (SEC), ion exchange chromatography (IEC), high performance liquid chromatography (HPLC), reversed phase HPLC (RP-HPLC), ultra performance liquid chromatography (UPLC), and reversed phase UPLC (RP-UPLC).

In a second aspect, the present invention provides a glucagon-like peptide-1 agonist peptide represented by formula (1),

(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH

wherein:
X is Ala or Aib;
R is -γ-Glu-Hexadecanoic acid or -AEEA-AEEA-γ-Glu-Octadecanedioic acid; and
a content of deletion of X at position 2 of the glucagon-like peptide-1 agonist peptide is less than 0.10%.

In some embodiments, the glucagon-like peptide-1 agonist peptide represented by formula (1), wherein the content of deletion of X at position 2 of the glucagon-like peptide-1 agonist peptide is less than 0.09%, preferably is less than 0.08%, most preferably is less than 0.07%.

In some embodiments, the glucagon-like peptide-1 agonist peptide represented by formula (1), wherein a content of deletion of Thr at position 5 of the glucagon-like peptide-1 agonist peptide is less than 0.06%, preferably is less than 0.05%.

In some embodiments, the glucagon-like peptide-1 agonist peptide represented by formula (1), wherein a content of deletion of Gly at position 4 of the glucagon-like peptide-1 agonist peptide is less than 0.01%.

In a third aspect, the present invention provides a glucagon-like peptide-1 agonist peptide obtained according to a process for the preparation of glucagon-like peptide-1 agonist peptide represented by formula (1),

(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH

wherein:
X is Ala or Aib;
R is -γ-Glu-Hexadecanoic acid or -AEEA-AEEA-γ-Glu-Octadecanedioic acid; comprising steps of:
(a) synthesizing a protected peptide on a solid phase by stepwise coupling of a protected amino acid and a protected dipeptide, the protected dipeptide comprising a pseudoproline dipeptide and a non-pseudoproline dipeptide;
(b) cleaving the protected peptide from the solid phase, and deprotecting the protected peptide to obtain the peptide;
a content of deletion of X at position 2 of the glucagon-like peptide-1 agonist peptide is less than 0.10%.

### IV. EXAMPLES

The purity and content of impurities were determined by HPLC for crude peptide and final peptide. Specifically, the impurity calculation was described as below. Each of peaks from the HPLC chromatogram were collected separately and totally injected into the HPLC-ESI (Electrospray Ionization)-MS system for mass identification. For HPLC-ESI-MS system, the reverse-phase columns packed with C18 matrix were used to bind peptides in high-aqueous mobile phase, salts and buffers were washed off, and the peptides were eluted using a high-organic mobile phase. In the TIC (total ion current) chromatogram, the signals other than blank would be identified and extracted to afford EIC (extracted-ion chromatogram). The intensity of each EIC would be counted as a ratio expressed as a fraction of 100 then multiplied by the RA % (relative area percent) of the peak in HPLC chromatogram where the sample collected from to afford the final value of the impurities. The Des-Aib² in the following mentioned Comparative Example 1 was used as an example:

| | | | Monoisotopic | Delta of | Relative | RA% |
|---|---|---|---|---|---|---|
| RRT | m/z | Charge | mass (Da) | Semaglutide | amount | of peak |
| 0.99 | 1337.7 | 3 | 4010.1 | −101.049 | 1 | 0.99% |
| | 1343.0 | 3 | 4026.1 | −85.055 | 0.36 | |
| | 1371.4 | 3 | 4111.1 | 0.001 | 0.30 | |
| | 1406.7 | 3 | 4217.2 | 106.034 | 0.30 | |
| | 1395.1 | 3 | 4182.2 | 71.071 | 0.11 | |

$Amount of Des - {Aib}^{2} = \frac{0.36 \times 0.99 \%}{1 + 0.36 + 0.3 + 0.3 + 0.11} = 0.17 \%$

The purity (peak area percent) of final peptide was evaluated by the following principle:
∘: Purity≥98%
Δ: 97%≤Purity<98%
×: 96%≤Purity<97%

The conversion rate was calculated by the following formula:

Conversion rate=[The weight of obtained final peptide]/[The weight of used blank resin]

The estimated total recovery was calculated by the following formula:

$$Estimated total recovery ( \% ) = \frac{Conversion rate \times peptide content \times 1000}{substitution degree of Wang resin \times MW of final peptide}$$

Peptide content[%(w/w)]: the final peptide subtracting the content of water and salt

### Comparative Example 1

Preparation of Fmoc-Gly-Wang Resin

30 g of dry Wang Resin (with a degree of substitution of 1.04 mmol/g) was weighed and added into a SPPS reactor. The resin was firstly washed twice with DMF, again swollen for 30 min with DMF in a volume 2-3 times the resin bed, and washed with DMF three times. 18.912 g of Fmoc-Gly-OH (2.0 equiv.) and 9.559 g of HOBt·H₂O (2.0 equiv.) were dissolved in a mixed solvent of DMF and DCM. After the amino acid was dissolved, the solution was poured into the SPPS reactor following by addition of 9.8 ml of DIC (2.0 equiv.) and 0.762 g of DMAP (0.2 equiv.). The resulting reaction mixture was stirred with nitrogen blowing at ambient temperature for 24 h. After the reaction was completed, an appropriate amount of a mixed solution of acetic anhydride and pyridine (volume ratio: Ac₂O/DIPEA=½) was added to block the reaction for 30 min or more, following washing three times with DMF shrinking twice with methanol, and drying under reduced pressure. After the reaction was stopped, Fmoc-Gly-Wang Resin with a degree of substitution of 0.999 mmol/g was obtained.

Synthesis of Peptide

25.869 g of Fmoc-Gly-Wang Resin (19.977 mmol) with a degree of substitution of 0.999 mmol/g was weighed and swelled for 30 min with DMF, followed by sequentially coupling according to the amino acid sequence of SEQ ID NO: 1 with 2-3 equivalents of amino acid feeds and condensing with DIC+A or B+C, until the resin was detected to be transparent with chloranil, wherein A was HOBt·H₂O or Ethyl (hydroxyimino)cyanoacetate; B was HATU or DEPBT; C was DIPEA; and the solvent was chosen from DMF or DCM. All amino acids coupled were commercially available Fmoc monoamino acids, except for the last His residue was coupled as a Boc-His(Boc)-OH. DCHA residue. Fmoc deprotection was achieved by 5% (w/w) piperidine in DMF followed by DMF washes to completely remove the base reagent. Washing efficiency was assessed by chloranil test, washing was repeated until no blue coloring could be observed any more prior to coupling. All couplings proceeded well and did not require re-coupling.

Before the coupling of the Lys side chain, Alloc protecting groups were removed with a solution of 0.2 to 0.3 equivalents of Pd(PPh₃)₄ and 10 equivalents of morpholine in DCM for 1 h. The coupling of the remaining amino acids on the side chain was completed at second time with the tBuOSteGlu(AEEA-AEEA)OtBu fragment. 5.1683 g of resin bound peptide was used, and around g of resin bound peptide was obtained after Alloc-deprotection and coupling completed.

32 mL cleaving agent was formulated in a volume ratio of TFA:EDT:PPW=90:13.6:5 was added into a 100 mL reaction kettle, and pre-cooling to 0-10° C. 4.00 g of resin bound peptide obtained above was taken and slowly added into the reaction kettle, allowing to react for 2 h at the room temperature. After the reaction was completed, the resin was filtered off and the filtrate was collected. The resin was washed with a small amount of TFA, and the filtrates were combined then cooled to 0-10° C. The ice-cold MTBE was added to the filtrate, the crude peptide was precipitated at 0-10° C., filtered, washed with MTBE, and dried under vacuum to obtain 2.29 g of crude peptide. The crude peptide was further purified by HPLC process disclosed in U.S. Patent Publication No. 20210206800 to obtain 550 mg of final peptide, semaglutide.

### Comparative Example 2

Synthesis of Peptide

6 g of Fmoc-20mer-Wang Resin (1.4 mmol) synthesized in Comparative Example 1 with a degree of substitution of 0.999 mmol/g was weighed and swelled for 30 min with DMF, followed by sequentially coupling according to the amino acid sequence of SEQ ID NO: 1 with 2-3 equivalents of amino acid feeds and condensing with DIC+A or B+C, until the resin was detected to be transparent with chloranil, wherein A was HOBt·H₂O or Ethyl (hydroxyimino)cyanoacetate; B was HATU or DEPBT; C was DIPEA; and the solvent was chosen from DMF or DCM. All amino acids coupled were commercially available Fmoc monoamino acids, except for the Val¹⁰-Ser¹¹ residue was coupled as a pseudoproline dipeptide Fmoc-Val-Ser(Ψ^{(Me,Me)}pro)-OH residue; the last His was coupled as a Boc-His(Boc)-OH. DCHA residue. Fmoc deprotection was achieved by 20% (w/w) piperidine in DMF followed by DMF washes to completely remove the base reagent. Washing efficiency was assessed by chloranil test, washing was repeated until no blue coloring could be observed any more prior to coupling. All couplings proceeded well and did not require re-coupling.

Before the coupling of the Lys side chain, Alloc protecting groups were removed with a solution of 0.2 to 0.3 equivalents of Pd(PPh₃)₄ and 10 equivalents of morpholine in DCM for 1 h. The coupling of the remaining amino acids on the side chain was sequentially coupled with the Fmoc-AEEA-OH (3.0 equiv.) twice and tBuSte-Glu-tBu.DCHA (3.0 equiv.) once and condensing with DIC+A or B+C, until the resin was detected to be transparent with chloranil, wherein A was HOBt·H₂O or Ethyl (hydroxyimino)cyanoacetate; B was HATU or DEPBT; C was DIPEA; and the solvent was chosen from DMF or DCM. Fmoc deprotection was achieved by 20% (w/w) piperidine in DMF followed by DMF washes to completely remove the base reagent. Washing efficiency was assessed by chloranil test, washing was repeated until no blue coloring could be observed any more prior to coupling. All couplings proceeded well and did not require re-coupling. 2.09 g of resin bound peptide was used, and around 2.21 g of resin bound peptide was obtained after Alloc-deprotection and coupling completed.

2.21 g of resin bound peptide obtained above was taken and added into a 50 mL reaction kettle. 17.84 mL of cleaving agent was formulated in a volume ratio of TFA:EDT:PPW=83:12.5:4.5, and pre-cooling to 0-10° C., and then poured into the 50 mL reaction kettle, allowing to react for 2 h at the room temperature. After the reaction was completed, the resin was filtered off and the filtrate was collected. The resin was washed with a small amount of TFA, and the filtrates were combined then cooled to 0-10° C. The ice-cold MTBE was added to the filtrate, the crude peptide was precipitated at 0-° C., filtered, washed with MTBE, and dried under vacuum to obtain 1.26 g of crude peptide. 900 mg of crude peptide was further purified by HPLC process disclosed in U.S. Patent Publication No. 20210206800 to obtain 319 mg of final peptide, semaglutide.

### Comparative Example 3

Synthesis of Peptide

6 g of Fmoc-20mer-Wang Resin (1.4 mmol) synthesized in Comparative Example 1 with a degree of substitution of 0.999 mmol/g was weighed and swelled for 30 min with DMF, followed by sequentially coupling according to the amino acid sequence of SEQ ID NO: 1 with 2-3 equivalents of amino acid feeds and condensing with DIC+A or B+C, until the resin was detected to be transparent with chloranil, wherein A was HOBt·H₂O or Ethyl (hydroxyimino)cyanoacetate; B was HATU or DEPBT; C was DIPEA; and the solvent was chosen from DMF or DCM. All amino acids coupled were commercially available Fmoc monoamino acids, except for the Val¹⁰-Ser¹¹ residue was coupled as a pseudoproline dipeptide Fmoc-Val-Ser(Ψ^{(Me,Me)}pro)-OH residue; the last His was coupled as a Boc-His(Boc)-OH. DCHA residue. Fmoc deprotection was achieved by 20% (w/w) piperidine in DMF followed by DMF washes to completely remove the base reagent. Washing efficiency was assessed by chloranil test, washing was repeated until no blue coloring could be observed any more prior to coupling. All couplings proceeded well and did not require re-coupling.

Before the coupling of the Lys side chain, Alloc protecting groups were removed with a solution of 0.2 to 0.3 equivalents of Pd(PPh₃)₄ and 10 equivalents of morpholine in DCM for 1 h. The coupling of the remaining amino acids on the side chain was completed at second time with the tBuOSteGlu(AEEA-AEEA)OtBu fragment. 5.1683 g of resin bound peptide was used, and around g of resin bound peptide was obtained after Alloc-deprotection and coupling completed.

2.21 g of resin bound peptide obtained above was taken and added into a 50 mL reaction kettle. 17.84 mL of cleaving agent was formulated in a volume ratio of TFA:EDT:PPW=83:12.5:4.5, and pre-cooling to 0-10° C., and then poured into the 50 mL reaction kettle, allowing to react for 2 h at the room temperature. After the reaction was completed, the resin was filtered off and the filtrate was collected. The resin was washed with a small amount of TFA, and the filtrates were combined then cooled to 0-10° C. The ice-cold MTBE was added to the filtrate, the crude peptide was precipitated at 0-10° C., filtered, washed with MTBE, and dried under vacuum to obtain 1.26 g of crude peptide. The crude peptide was further purified by HPLC process disclosed in U.S. Patent Publication No. 20210206800 to obtain 400 mg of final peptide, semaglutide.

### Example 1

Preparation of Fmoc-Gly-Wang Resin

40 g of dry Wang Resin (with a degree of substitution of 1.07 mmol/g) was weighed and added into a SPPS reactor. The resin was firstly washed twice with DMF, again swollen for 30 min with DMF in a volume 2-3 times the resin bed and washed with DMF three times. 38.18 g of Fmoc-Gly-OH (3.0 equiv.) and 19.667 g of HOBt·H₂O (3.0 equiv.) were dissolved in DMF. After the amino acid was dissolved, the solution was poured into the SPPS reactor following by addition of 20 ml of DIC (3.0 equiv.) and 1.5693 g of DMAP (0.3 equiv.). The resulting reaction mixture was stirred with nitrogen blowing at ambient temperature for 16 h. After the reaction was completed, an appropriate amount of a mixed solution of acetic anhydride and pyridine (volume ratio: Ac₂O/DIPEA=½) was added to block the reaction for 30 min or more, following washing three times with DMF shrinking twice with methanol, and drying under reduced pressure. After the reaction was stopped, Fmoc-Gly-Wang Resin with a degree of substitution of 1.061 mmol/g was obtained.

Synthesis of Peptide

39.433 g of Fmoc-Gly-Wang Resin (31.8 mmol) with a degree of substitution of 1.061 mmol/g was weighed and swelled for 30 min with DMF, followed by sequentially coupling according to the amino acid sequence of SEQ ID NO: 1 with 2-3 equivalents of amino acid feeds and condensing with DIC+A or B+C, until the resin was detected to be transparent with chloranil, wherein A was HOBt·H₂O or Ethyl (hydroxyimino)cyanoacetate; B was HATU or DEPBT; C was DIPEA; and the solvent was chosen from DMF. All amino acids coupled were commercially available Fmoc monoamino acids, except for the Val¹⁰-Ser¹¹ residue was coupled as a pseudoproline dipeptide Fmoc-Val-Ser(Ψ^{(Me,Me)}pro)-OH residue and condensing with DIC+Ethyl (hydroxyimino)cyanoacetate; the last His¹-Aib² was coupled as a Boc-His(Trt)-Aib-OH residue and condensing with DEPBT+DIPEA. Fmoc deprotection was achieved by 20% (w/w) piperidine in DMF followed by DMF washes to completely remove the base reagent. Washing efficiency was assessed by chloranil test, washing was repeated until no blue coloring could be observed any more prior to coupling. All couplings proceeded well and did not require re-coupling.

Before the coupling of the Lys side chain, Alloc protecting groups were removed with a solution of 0.05 to 0.1 equivalents of Pd(PPh₃)₄ and 10 equivalents of morpholine in Toluene for 6 h. The coupling of the remaining amino acids on the side chain was completed at second time with the tBuOSteGlu(AEEA-AEEA)OtBu fragment. Around 26.11 g of resin bound peptide was obtained after Alloc-deprotection and coupling completed.

173 mL of cleaving agent was formulated in a volume ratio of TFA:EDT:PPW=90:13.6:5 was added into a 500 mL reaction kettle, and pre-cooling to 0-10° C. 25.213 g of resin bound peptide obtained above was taken and slowly added into the reaction kettle, allowing to react for 6 h at 0-10° C. After the reaction was completed, the resin was filtered off and the filtrate was collected. The resin was washed with a small amount of TFA, and the filtrates were combined then cooled to −30-0° C. The ice-cold MTBE was added to the filtrate, the crude peptide was precipitated at −30-0° C., filtered, washed with MTBE, and dried under vacuum to obtain 14.229 g of crude peptide. The crude peptide was further purified by HPLC process disclosed in U.S. Patent Publication No. 20210206800 to obtain 5.84 g of final peptide, semaglutide.

The analytical results of Example 1 and Comparative Example 1-3 were summarized in Table 3.

TABLE 3

| | | Example | Comparative Example | | |
|---|---|---|---|---|---|
| | | 1 | 1 | 2 | 3 |
| Pseudoproline dipeptide | | ∘ | x | ∘ | ∘ |
| (Fmoc-Val-Ser(Psi^{(Me, Me)}pro)- | | | | | |
| OH) | | | | | |
| Non-pseudoproline dipeptide | | ∘ | x | x | x |
| (Boc-His(Trt)-Aib-OH) | | | | | |
| R | Sequentially | | | ∘ | |
| | coupled | | | | |
| | Full side | ∘ | ∘ | | ∘ |
| | chain coupled | | | | |

Analytical result

| Purity of final peptide | | ∘ | x | Δ | ∘ |
|---|---|---|---|---|---|
| Critical | Des-Aib² | <0.05% | 0.17% | 0.10% | 0.11% |
| impurity | Des-Thr⁵ | 0.04% | 0.50% | 0.07% | 0.06% |
| | Des-Gly⁴ | <0.01% | 0.08% | 0.01% | 0.01% |

| | | | | |
|---|---|---|---|---|
| Conversion rate | 1.21 | 0.78 | 0.80 | 0.89 |
| [final peptide/blank resin] | | | | |
| Estimated total recovery | 24.7% | 16.9% | 17.3% | 22.0% |

Des-Aib²: the impurity that is a deletion of Aib at position 2 of semaglutide.
Des-Thr⁵: the impurity that is a deletion of Thr at position 5 of semaglutide.
Des-Gly⁴: the impurity that is a deletion of Gly at position 4 of semaglutide.

Based on comparing the analytical results of Example 1 and Comparative Example 1-3, the pseudoproline dipeptide, such as Fmoc-Val-Ser(Psi^{(Me,Me)}pro)-OH and the non-pseudoproline dipeptide, such as Boc-His(Trt)-Aib-OH, which are used together in the synthesis of semaglutide, have the synergistic effect that can dramatically reduce the Des-Thr⁵ and Des-Aib² impurities and increase the yield (conversion rate and estimated total recovery). And based on comparing the analytical results of Comparative Example 2-3, the full side chain process is better than sequentially coupled side chain process.

### Example 2

The synthetic process was similar to the description in Example 1.

### Example 3

The synthetic process was similar to the description in Example 1, except for Boc-His(Boc)-Aib-OH was used to replace Boc-His(Trt)-Aib-OH.

### Example 4

The synthetic process was similar to the description in Example 1, except for the side chain was sequentially coupled and similar to the description in Comparative Example 3.

### Example 5

The synthetic process was similar to the description in Example 1, except for Boc-His(Boc)-Aib-OH was used to replace Boc-His(Trt)-Aib-OH, and the side chain was sequentially coupled.

The analytical results of Example 2-5 were summarized in Table 4.

TABLE 4

| | | Example | | | |
|---|---|---|---|---|---|
| | | 2 | 3 | 4 | 5 |
| Pseudoproline dipeptide | | ∘ | ∘ | ∘ | ∘ |
| (Fmoc-Val-Ser(Psi^{(Me, Me)}pro)-OH) | | | | | |
| Non-pseudoproline | Boc-His(Trt)-Aib-OH | ∘ | x | ∘ | x |
| dipeptide | Boc-His(Boc)-Aib-OH | x | ∘ | x | ∘ |
| R | Sequentially coupled | | | ∘ | ∘ |
| | Full side chain coupled | ∘ | ∘ | | |

Analytical result

| | | | | |
|---|---|---|---|---|
| Purity of crude peptide | 34.85% | 34.51% | 35.8% | 29.4% |

Based on comparing the analytical results of the purity of crude peptide from Example 2-5, Boc-His(Trt)-Aib-OH) is better than Boc-His(Boc)-Aib-OH) in the synthesis of semaglutide, either full side chain process or sequentially coupled side chain process.

### Example 6

The synthetic process was similar to the description in Example 1.

### Example 7

The synthetic process was similar to the description in Example 1, except for HATU/DIPEA was used as the coupling reagent.

### Example 8

The synthetic process was similar to the description in Example 1, except for DIC was used as the coupling reagent.

The analytical results of Example 6-8 were summarized in Table 5.

TABLE 5

| | | Example | | |
|---|---|---|---|---|
| | | 6 | 7 | 8 |
| Pseudoproline dipeptide | | ∘ | ∘ | ∘ |
| (Fmoc-Val-Ser(Psi^{(Me, Me)}pro)-OH) | | | | |
| Non-pseudoproline dipeptide | | ∘ | ∘ | ∘ |
| (Boc-His(Trt)-Aib-OH) | | | | |
| Coupling reagent | DEPBT/DIPEA | ∘ | x | x |
| | HATU/DIPEA | x | ∘ | x |
| | DIC | x | x | ∘ |
| R | Sequentially coupled | | | |
| | Full side chain coupled | ∘ | ∘ | ∘ |

Analytical result

| | | | |
|---|---|---|---|
| Purity of crude peptide | 60.35% | 52.17% | 57.13% |
| Purity of final peptide | ∘ | ∘ | ∘ |
| Conversion rate | 1.21 | 0.84 | 0.32 |
| [final peptide/blank resin] | | | |

Based on comparing the analytical results of the purity of crude peptide and conversion rate from Example 6-8, the coupling reagent DEPBT/DIPEA is better than HATU/DIPEA or DIC in the coupling of non-pseudoproline dipeptide Boc-His(Trt)-Aib-OH.

The invention is not limited by the embodiments described above which are presented as examples only but can be modified in various ways within the scope of protection defined by the appended patent claims.

What is claimed is:

1. A process for the preparation of glucagon-like peptide-1 agonist peptide represented by formula (1),
(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH
wherein:
X is Ala or Aib;
R is -γ-Glu-Hexadecanoic acid or -AEEA-AEEA-γ-Glu-Octadecanedioic acid;
comprising steps of:
(a) synthesizing a protected peptide on a solid phase by stepwise coupling of a protected amino acid and a protected dipeptide, the protected dipeptide comprising a pseudoproline dipeptide and a non-pseudoproline dipeptide;
(b) cleaving the protected peptide from the solid phase, and deprotecting the protected peptide to obtain the peptide.

2. The process according to claim 1, wherein the pseudoproline dipeptide is Fmoc-Val-Ser(Psi^{(Me,Me)}pro)-OH.

3. The process according to claim 1, wherein the non-pseudoproline dipeptide is selected from the group consisting of Boc-His(Boc)Aib-OH, Boc-His(Boc)Ala-OH, Boc-His(Trt)Aib-OH, and Boc-His(Trt)Ala-OH.

4. The process according to claim 3, wherein the non-pseudoproline dipeptide further comprises at least one selected from the group consisting of Boc-Arg(Pbf)Gly-OH and Boc-Glu(OtBu)Gly-OH.

5. The process according to claim 4, wherein the non-pseudoproline dipeptide comprises no more than 3 types of dipeptide.

6. The process according to claim 3, wherein the non-pseudoproline dipeptide is selected from the group consisting of Boc-His(Trt)Aib-OH and Boc-His(Trt)Ala-OH.

7. The process according to claim 6, wherein the coupling of the non-pseudoproline dipeptide with the protected amino acid is carried out in presence of a coupling reagent consisting of DEPBT and DIPEA.

8. The process according to claim 1, wherein the protected amino acid is Fmoc-Lys(Alloc)-OH or Fmoc-Lys(ivDde)-OH.

9. The process according to claim 1, wherein the peptide is liraglutide.

10. The process according to claim 1, wherein the peptide is semaglutide.

11. The process according to claim 10, wherein the R is -AEEA-AEEA-γ-Glu-Octadecanedioic acid, and the process comprises coupling AEEA-AEEA-(γ-Glu-OtBu)-Octadecanedioic acid mono-tert-butyl ester with ε-amino side chain of Lys after the step (a).

12. The process according to claim 10, wherein the R is -AEEA-AEEA-γ-Glu-Octadecanedioic acid, and the process comprises sequentially coupling Fmoc-AEEA-OH, Fmoc-AEEA-OH, (γ-Glu-OtBu)-Octadecanedioic acid mono-tert-butyl ester with ε-amino side chain of Lys after the step (a).

13. The process according to claim 1 further comprising a step of purification after the step (b).

14. A glucagon-like peptide-1 agonist peptide represented by formula (1),
(1)
H-His¹-X²-Glu³-Gly⁴-Thr⁵-Phe⁶-Thr⁷-Ser⁸-Asp⁹-Val¹⁰-
Ser¹¹-Ser¹²-Tyr¹³-Leu¹⁴-Glu¹⁵-Gly¹⁶-Gln¹⁷-Ala¹⁸-Ala¹⁹-
Lys²⁰(R)-Glu²¹-Phe²²-Ile²³-Ala²⁴-Trp²⁵-Leu²⁶-Val²⁷-
Arg²⁸-Gly²⁹-Arg³⁰-Gly³¹-OH
wherein:
X is Ala or Aib;
R is -γ-Glu-Hexadecanoic acid or -AEEA-AEEA-γ-Glu-Octadecanedioic acid; and
a content of deletion of X at position 2 of the glucagon-like peptide-1 agonist peptide is less than 0.10%.

15. The glucagon-like peptide-1 agonist peptide according to claim 14, wherein a content of deletion of Thr at position 5 of the glucagon-like peptide-1 agonist peptide is less than 0.06%.

16. The glucagon-like peptide-1 agonist peptide according to claim 14, wherein a content of deletion of Gly at position 4 of the glucagon-like peptide-1 agonist peptide is less than 0.01%.

17. A glucagon-like peptide-1 agonist peptide obtained according to a process of claim 1, wherein a content of deletion of X at position 2 of the glucagon-like peptide-1 agonist peptide is less than 0.10%.

18. The glucagon-like peptide-1 agonist peptide according to claim 17, wherein a content of deletion of Thr at position 5 of the glucagon-like peptide-1 agonist peptide is less than 0.06%.

19. The glucagon-like peptide-1 agonist peptide according to claim 18, wherein a content of deletion of Gly at position 4 of the glucagon-like peptide-1 agonist peptide is less than 0.01%.
